(19) United States
(12) Patent Application Publication
(10) Pub. No.: US 2009/0105574 A1
(43) Pub. Date: Apr. 23, 2009

(54) MAGNETIC ELECTRODE DEVICE AND AN ELECTROCARDIOGRAPH DETECTING METHOD THEREOF

(75) Inventor: Shuenn-Tsong Young, Taipei City (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL YANG-MING UNIVERSITY, Taipei City (TW)

(21) Appl. No.: 11/877,098

(22) Filed: Oct. 23, 2007

(51) Int. Cl. A61B 5/0408

(52) U.S. Cl. 600/372; 600/509

(57) ABSTRACT

A magnetic electrode device and it further provides a method of CEG detection and record by the use of the said electrode device.

### FIELD OF THE INVENTION

The present invention relates to a device for detecting electrocardiograph (ECG) signals and a method thereof. The electrode device comprises an electrode and a supporting device thereof. In addition, the present invention also provides a method for detecting ECG using said magnetic electrodes, conducting wires and a portable ECG detecting machine.

### BACKGROUND

Unlike skeletal muscle, which contracts in response to nerve stimulation, and like single unit smooth muscle, cardiac muscle is myogenic, meaning that it is self-excitable stimulating contraction without a requisite electrical impulse coming from the central nervous system.

A single cardiac muscle cell, if left without input, will contract rhythmically at a steady rate; if two cardiac muscle cells are in contact, whichever one contracts first will stimulate the other to contract, and so on. This inherent contractile activity is heavily regulated by the autonomic nervous system. If synchronization of cardiac muscle contraction is disrupted for some reason, for example, in a heart attack, uncoordinated contraction known as fibrillation can result.

With each heart beat, an electrical impulse travels through the heart. This impulse causes the heart muscle to squeeze and pump blood from the heart. ECG (electrocardiograph) is a record of the electrical activity of cardiac tissues. An ECG is obtained by measuring electrical potential between various points of the body using a biomedical instrumentation amplifier. Analysis of the various waves and normal vectors of depolarization and repolarization yields important diagnostic information.

Generally, if a patient needed an ECG, he or she had to lie down next to a huge ECG machine in the hospital rather than record it at home or carry an ECG detecting machine with him or her. However, many cardiac patients did not have irregular heart beats all the time. It was possible that they had regular heart beats when having their ECGs recorded in hospitals, so the diagnosis might not be accurate due to the complex condition of cardiac patients. If the patients use a portable or prolonged recording ECG machine, they would get more instantaneously ECG.

Nowadays, there are many different kinds of electrode for ECG, and among them, the mostly used are sticky pads and dry electrodes with special band, like the one disclosed in Taiwan Patent No: M262157 (incorporated herein by reference in its entirety). However, those electrodes are not suitable for prolonged use. For example, the sticky pads might cause users itch when pasted on the skin for a long time, and its stickiness would be reduced overtime. As for dry electrode, users have to wear special bands to hold the electrodes on the skin. The bands allow users to fix the electrode on the skin for a long time, but it's very uncomfortable and unaesthetic. Besides, users can not adjust the relative position of electrode according the state they are in; therefore, they can't obtain the best eletrophysical signal when they are under different conditions.

### SUMMARY OF THE INVENTION

The present invention relates to a magnetic electrode device, comprising a magnetic electrode and a buckle attached with a magnet, wherein the buckle can attract the corresponding electrode. The magnetic electrode comprises an electrode and magnetic material, wherein the magnetic electrode is attached to the electrode. The present invention also relates a method of detecting ECG (electrocardiograph), comprising the following steps: provide a magnetic electrode device of claim 1; place the electrode on a user's skin; hook or clip the buckle on cloth or accessories near the skin, so the buckle can automatically attract the corresponding electrode; carry a portable ECG detecting machine; and then attach one end of the conducting wire on the electrode and the other end on the portable ECG detecting machine.

### BRIEF DESCRIPTION OF DRAWING

FIG. 1 shows the corresponding position of electrode device, skin and belt.
10: A magnetic electrode device;
20: A magnetic buckle;
21: A buckle;
22: magnetic material;
23: A belt;
24: cloth
30: A magnetic electrode;
32: A dry electrode;
33: skin.
40: A conducting wire

FIG. 2 shows the use of electrode device as an ECG detecting apparatus.

### DETAIL DESCRIPTION OF THE INVENTION

The present invention provides a magnetic electrode device and an electrocardiograph detecting method by the use of the magnetic electrode device. The magnetic electrode device comprises a dry electrode and magnetic buckle wherein the dry electrode is pasted with magnetic material and the buckle can be hooked on a belt, a bra or tights, and it can attract the corresponding electrode as well. The present invention has advantages over the prior art because the electrode doesn't need to be fixed or supported by special band. Users can simply fix the magnetic buckle on their belts or tights and use their belts or tights as supports to immobilize the electrode. Therefore, the dry electrode can be attached properly on the skin and provide continuous detecting of electrophysical signals. The present invention also relates to a method for detecting ECG using the said magnetic electrode device, conducting wire and portable

A user can move the buckle along the belt and the corresponding electrode will be moved accordingly to an appropriate place. The present invention provides users with a convenient and artistic detecting device. In addition, the present invention also provides a method for detecting electrocardiograph using magnetic electrodes, conducting wire and a portable ECG detecting machine. Users do not have to wear additional band and they can adjust the position of electrode easily to obtain the best signal. It's very convenient, practical and aesthetic.

While the above description contains many specificities, these should not be constructed as limitation on the scope of the invention, but rather as an exemplification of one preferred embodiment thereof. Many other variations are possible. Accordingly, the scope of the invention should be determined not by the embodiments illustrated, but by the appended claims and their legal equivalents.

### EXAMPLES

### Example 1

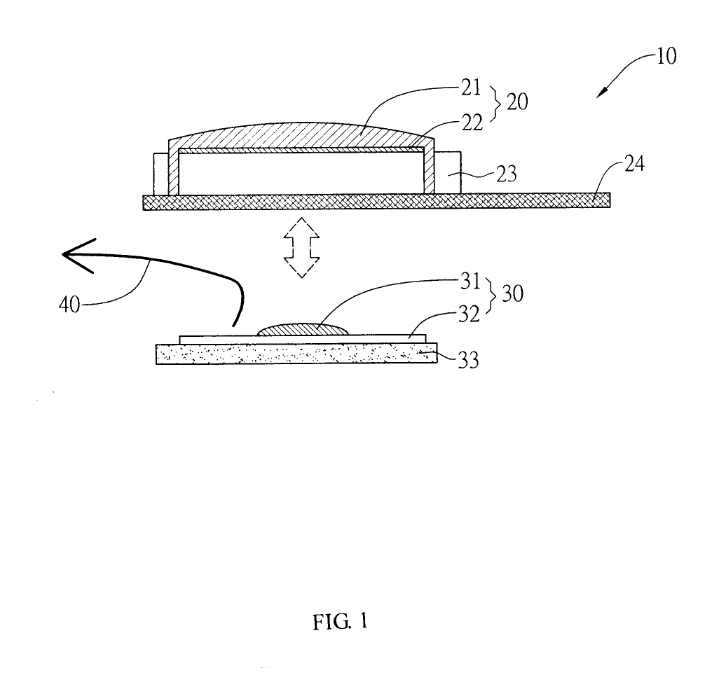

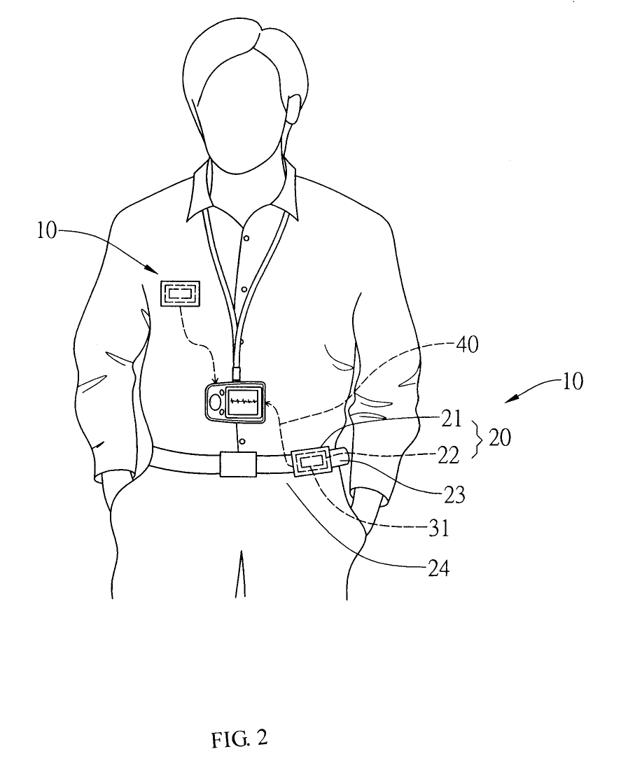

The present invention uses dry electrode to detect ECG signal, wherein the dry electrode is attached with magnetic material which can be attracted by the corresponding magnetic buckle hooked on the user's belt. The dry electrode with the magnetic material attached on touches the skin directly and the magnetic force of the magnetic buckle attracts it regardless the clothes. Since the buckle attracts the electrode, the belt on which the buckle hooks provides a support for the electrode, so that the electrode can be fixed on the skin properly. FIG. 1 shows the corresponding position of electrode device, skin and belt; while FIG. 2 shows the use of electrode device as an ECG detecting apparatus.

### Example 2

The present invention uses dry electrode to detect ECG signals wherein the dry electrode is not fixed on the skin by special band; instead, it is attached with magnetic material which can be attracted by the corresponding magnetic buckle. This magnetic buckle can be clipped on the steel brace of bra. The dry electrode directly touches the skin and the corresponding magnetic buckle can attracts the magnetic material attached on the back of the dry electrode regardless of the cloth. The bra can provide dry electrode with a support, so that it can be fixed on the skin properly. The use of this electrode device as an ECG detecting apparatus is described as follows. The magnetic buckle is clipped on the steel brace of the bra while the dry electrode is placed on the skin and it is fixed on the skin by the magnetic force and the supporting of the bra. One end of the conducting wire is attached on the dry electrode while the other reaches out of the cloth and is connected to the portable ECG machine. Furthermore, the magnetic buckle can be clipped on any part of the bra where it attaches to the skin tightly to provide the dry electrode with support.

### Example 3

The present invention uses dry electrode as a constant ECG signal detecting device. The dry electrode touches the skin directly and it is attached with magnetic material on the back. The magnetic buckle is attached to the tights and provides magnetic force to attract the electrode. The tights help to fix the electrode on the skin properly. Moreover, the present invention also provides a method to detect ECG signals constantly, conveniently, aesthetically. A user can clip the magnetic buckle on his or her tights and magnetic forces helps to fix the electrode properly on the skin while he or she is exercising. One end of the conducting wire is attached to the electrode and the other end is connected to the portable ECG detecting machine. When everything is set up, the user can record his or her eletrophysical condition while exercising easily. This present invention can reduce the uncomfortable feeling when using a sticky pad all day long and the trouble of using a special band to support the dry electrode.

What is claimed is:

1. A magnetic electrode device, comprising:
(1) a magnetic electrode which comprises a electrode and magnetic material, wherein the magnetic electrode is attached to the electrode; and
(2) a buckle which is attached with magnet, wherein the magnet can attract the magnetic material on the electrode.

2. The device of claim 1, wherein the electrode is a dry electrode which can be placed on the skin to detect eletrophysical signals and be attached with magnetic material on the back.

3. The device of claim 1, wherein the buckle can be hooked on a belt, clipped on the brace of bra or on tights.

4. The device of claim 1, wherein the magnet attached to the interior of the buckle can attract the said electrode regardless of cloths, and the said electrode can be fixed on the skin properly by the support of the next-to-skin cloth or accessories.

5. The device of claim 1, it further comprises a conducting wire, wherein the wire can be connected to the electrode and used to conducting eletrophysical signals.

6. The device of claim 5, wherein the conducting electrode can be connected to any electrical device.

7. The device of claim 6, wherein the electrical device is portable ECG detecting machine.

8. A method of detecting ECG (electrocardiograph), comprising:
(1) the said magnetic electrode device of claim 1;
(2) place the said electrode on user's skin;
(3) hook or clip the said buckle on next-to-skin cloth or accessories, and the buckle can attract the corresponding electrode by the magnet attached to the interior of it
(4) carry a portable ECG detecting machine.
(5) attach one end of the conducting wire on the electrode and the other end on the portable ECG detecting machine.

9. The method of claim 8, wherein the buckle can be moved easily and frequently so that the corresponding electrode can be fixed on different part of skin to obtain the optical signals.

10. The method of claim 8, wherein one end of the conducting wire is attached on the corresponding electrode and the other reaches out of the cloth to be connected with the portable ECG detecting machine.

11. The method of claim 8, wherein the portable ECG detecting machine can be hung on user's neck, hooked on a belt or any possible ways of carrying.
